(19) United States
(12) Patent Application Publication
(10) Pub. No.: US 2022/0040347 A1
(43) Pub. Date: Feb. 10, 2022

(54) System and method for cleaning and disinfecting shopping carts

(71) Applicant: Mohamed Ferid Belkadhi, Montreal (CA)

(72) Inventor: Mohamed Ferid Belkadhi, Montreal (CA)

(21) Appl. No.: 17/444,786

(22) Filed: Aug. 10, 2021

Related U.S. Application Data
(60) Provisional application No. 63/063,474, filed on Aug. 10, 2020.

(51) Int. Cl. A61L 2/12; A61L 2/10; A61L 2/07; A61L 2/28; A61L 2/00; H05B 6/80; B08B 3/02; B08B 13/00; B62B 3/14

(52) U.S. Cl. CPC A61L 2/12; B08B 2203/0211; A61L 2/07; A61L 2/28; A61L 2/0047; H05B 6/80; B08B 3/022; B08B 13/00; B62B 3/14; A61L 2202/17; A61L 2202/122; A61L 2202/11; A61L 2202/14; A61L 2202/15; B08B 2203/007; A61L 2/10

(57) ABSTRACT

The present invention provides a system and method for cleaning and disinfecting shopping carts. The system comprises a main housing with three main systems: a steam system, an ultraviolet system and a microwave system. The combination of the systems allows for the reduction of germs and viruses without the use of chemicals which may be both harmful to the shopping carts and to the environment. A conveying means allows shopping carts to be displaced through the system. The system further provides a disinfecting box in connection with the main housing for users to disinfect their hands. The method provided allows for the cleaning and disinfecting of a shopping cart with the use of the system.

### CROSS-REFERENCE TO RELATED APPLICATIONS

The present patent application claims the benefits of priority of commonly assigned American Provisional Patent Application no. 63/063,474, entitled “CART DISINFECTION SYSTEM AND METHOD” and filed at the United States Patent and Trademark Office on Aug. 10, 2021.

### FIELD OF THE INVENTION

The present invention generally relates to a system and method for cleaning and disinfecting shopping carts and similar objects.

### BACKGROUND OF THE INVENTION

In certain settings, objects and/or surfaces may be subjected to repeated contact with different unwanted substances and particles. For example, shopping carts used in malls or groceries can be affected by germs, dirt, food or any kind of substances when in use and when stored. Unwanted substances may be acquired, among other things, when stored outside, when in use by the users or when in contact with food or beverages. For example, germs and virus may gather on shopping carts due to animals and insects when stored outside, with bird excrements being a common occurrence and being a propagation tool of avian influenza. Unclean shopping carts may be a source of discomfort for users, both visually and physically. Most of all, unclean shopping carts may be a source of disease and may transmit germs and viruses when not disinfected properly between each use.

With certain viruses spreading world-wide, such as the flu or a coronavirus, which may spread even when people are asymptomatic, it is especially essential for shopping carts to be properly cleaned and disinfected as they may be a significant cause of spread between people. There are generally two types of systems to clean and disinfect shopping carts, the first being manual and the second being automated.

Manual cleaning systems generally comprise an employee with a disinfecting solution and a cloth washing carts handles before or after each use. The drawbacks of this system is that it may be costly in the long run to constantly pay someone whenever the store is open, the employee cleaning and disinfecting the carts may get exposed to harmful particles, the amount of disinfecting solution used varies per each employee thus making it hard to plan accordingly, only the handles of the cart are generally cleaned while the rest of the cart may remain dirty and certain parts of the cart and of the handle of said cart may not be cleaned and disinfected equally. While manual systems may be sufficient for short term needs, they are to be avoided when the cleaning and disinfecting operation is to last for a long period of time.

Automated cleaning systems generally comprise either direct cleaning apparatus or indirect cleaning apparatus.

Direct cleaning apparatus are often automated brushes that physically clean a designated zone. A drawback of direct cleaning apparatus is that it may be hard for brushes to access all the surfaces of a cart since each model may have varying and irregular shapes. Another drawback of this system is that the cleaning apparatus may cause wear, damage or even destroy the cleaned object over time. Indeed, the brush may damage a protective coating on a cart handle over time. Yet another drawback of direct cleaning apparatus is that it often requires a lot of space for both the cleaning pieces to move and perform and for the mechanism allowing such movements. As in all physical systems, especially dynamic ones, direct cleaning apparatus may be prone to problems requiring maintenance or repairs, thus costing time and money.

Indirect cleaning apparatus usually rely on a high pressure spray and on chemical solutions to effectively clean and disinfect carts. In order to be effective at disinfecting, the sprayed solution requires a high concentrations of chemicals. The issue with using strong chemicals is that not only can they harm the materials of the shopping carts, but they may also harm the environment when drained out of the cleaning system and into wastewater drains. More so, the high pressure of the sprays used can be a source of unwanted noise and can require a lot of energy consumption.

Automated cleaning systems also generally lack sufficient safety systems in order to detect the presence of a child or an animal in the system before or during operations in order to prevent any accident. Indeed, shopping carts cleaning systems are often located in places wherein children or small animals may be present. In the case of children, parents may often be distracted due to varying elements related to the act of shopping and may not be able to properly avoid the entry of said children into the cleaning systems which may also happen very quickly. Usually, the entry points of said systems may be dimensioned big enough to allow the entry of said living beings and it may thus be a great risk and danger, especially considering the possible chemicals used, the heat and the electromagnetic waves present in such systems.

One way to help keep shopping carts clean and germ free is by having users wash and disinfect their own hands before using said shopping carts since users themselves are a source of germs. Indeed, multiple stores offer users cleaning systems for their hands. Those systems are generally independent from the shopping cart cleaning and disinfecting systems and thus require their own maintenance and space. Furthermore, those systems often distribute foam or fluids that comprise strong chemicals, thus making them harmful for the environment and for users' skin and even possibly health. It would therefore be quite helpful to have a system for disinfecting users' hands that is configured to work with a shopping cart cleaning and disinfecting system while not being harmful to the environment and to said users.

There is thus a need for a system and method that allows the efficient and safe cleaning and disinfecting of shopping carts while being economically and environmentally respectful.

### SUMMARY OF THE INVENTION

The aforesaid and other objectives of the present invention are realized by generally providing a system and method for cleaning and disinfecting a shopping cart, the system comprising a main housing having an entry point and an exit point; a conveying mean configured to displace the shopping cart from the entry point to the exit point; a steam system configured to emit steam in the main housing; an ultraviolet system configured to emit ultraviolet light within the main housing; and a microwave system configured to emit microwaves within the main housing, wherein the main housing comprises a section with holes for emitting microwaves.

The system may further comprise a disinfecting box, wherein the ultraviolet system is further configured to emit ultraviolet light in the disinfecting box. The conveying means may be a conveyor. The system may further comprise a fluid discharge system for the discharge of accumulated fluids inside the main housing. The system may yet further comprise visual markers showing the progress of the cleaning and disinfecting of the shopping cart. In another aspect of the invention, the entry and exit points may comprise curtains, the curtains being hermetic when closed. In yet another aspect of the invention, the system may further comprise an air valve to discharge air present in the main housing.

Further provided may be a method for cleaning and disinfecting a shopping cart, the method comprising the steps of: positioning a shopping cart in connection with a conveying mean so that it may be moved without manual interference; advancing the shopping cart to a first sensing element; opening an entry point; advancing the cart to a stationary position in a main housing; hermetically closing the entry point; activating a steam system; activating a microwave system; activating an ultraviolet system; showing the progress of a cleaning and disinfecting predetermined sequence to users with visual markers; opening an exit point, advancing the cart out of the main housing; and closing the exit point once a sensing element located at the exit point does not sense the presence of an object.

The method may further comprise discharging air present in the main housing by activating an air valve. The method may further comprise activating the microwave system after activating the steam system. The method may further comprise activating the microwave system for a period of 30 to 90 seconds. The method may further comprise discharging microwaves through a holed section of the main housing and discharging fluids accumulated in the main housing through a fluid discharge system. The method may further comprise cleaning and disinfecting a plurality of stacked shopping carts instead of a single shopping cart. The method may further comprise stopping any on-going operation when an emergency sequence is activated.

The features of the present invention which are believed to be novel are set forth with particularity in the appended claims.

### BRIEF DESCRIPTION OF THE DRAWINGS

The above and other objects, features and advantages of the invention will become more readily apparent from the following description, reference being made to the accompanying drawings in which:

FIG. 1 is an illustration of a front view of a first embodiment of a cleaning and disinfecting system in accordance with the principles of the present invention.

FIG. 2 is an illustration of a rear view from the embodiment of FIG. 1.

FIG. 3 is an illustration of a side view of the embodiment of FIG. 1.

FIG. 4 is an illustration of a top view of the embodiment of FIG. 1.

FIG. 5 is an illustration of a sectional view from the embodiment of FIG. 1.

FIG. 6 is an illustration of a sectional view from the embodiment of FIG. 3.

### DETAILED DESCRIPTION OF THE PREFERRED EMBODIMENT

A novel system and method for cleaning and disinfecting shopping carts will be described hereinafter. Although the invention is described in terms of specific illustrative embodiment(s), it is to be understood that the embodiment(s) described herein are by way of example only and that the scope of the invention is not intended to be limited thereby.

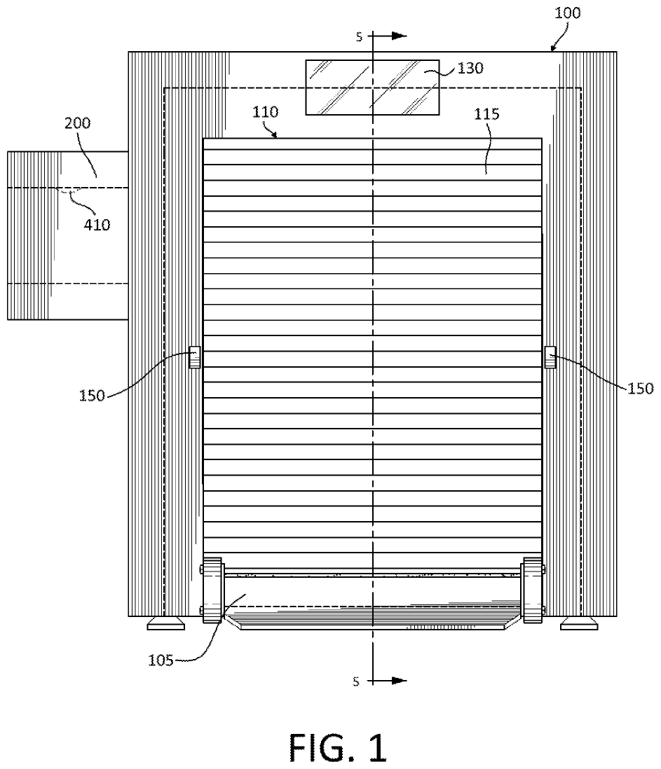

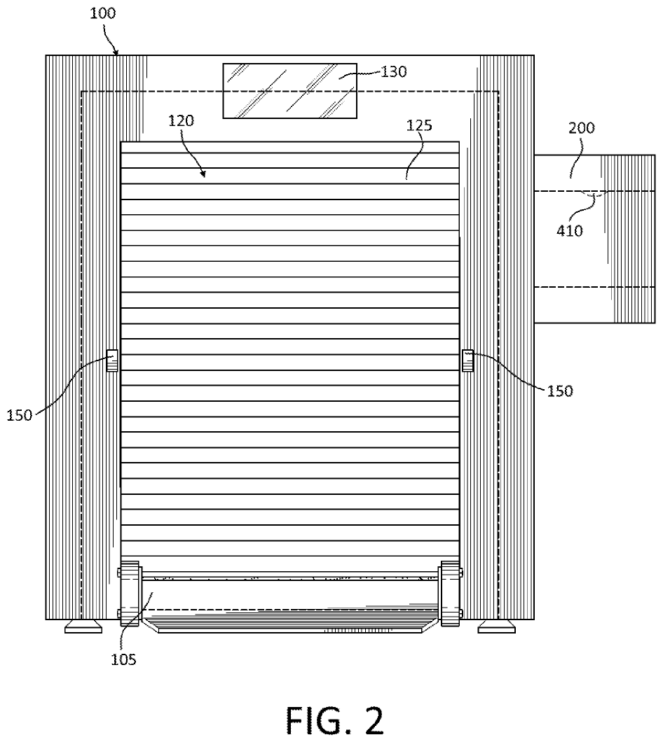

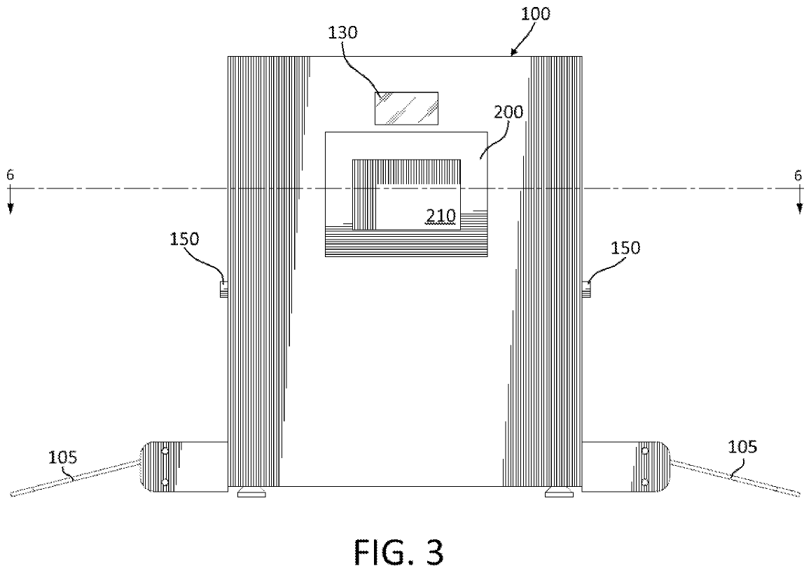

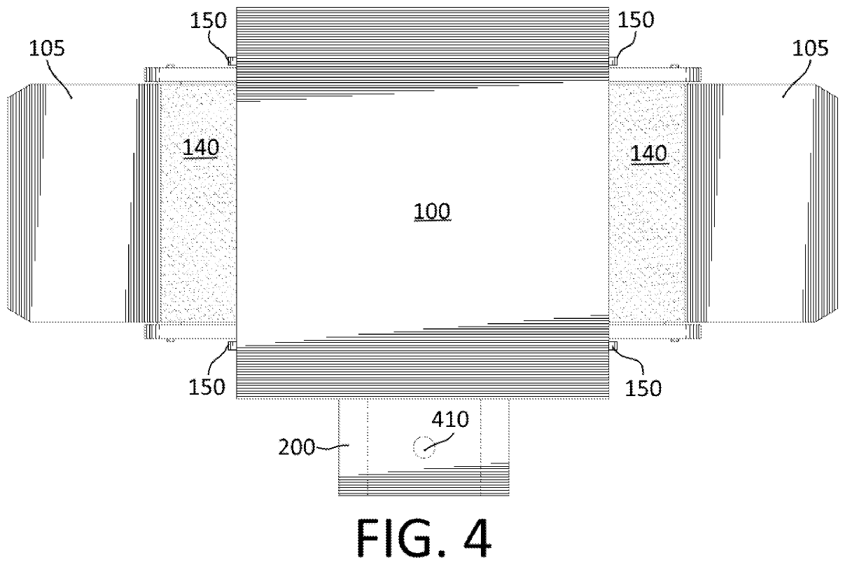

A first embodiment of the cleaning and disinfecting system may be seen in FIGS. 1 to 4. The system comprises a main housing 100 with an entry and an exit point (110, 120). The system may be elevated from the ground and elevation means 105 may be installed from and out of the entry and exit points (110, 120) to guide shopping carts into said system. The main housing 100 may have varying dimensions. In some embodiments, it may have dimensions to allow the presence of one shopping cart wherein other embodiments it may have dimensions to allow the presence of multiple stacked shopping carts. An external smaller housing 200 may further be located on an external surface of the main housing 100. The smaller housing 200, also referred to as disinfecting box, has an opening 210 wide enough to allow users to pass their hands through without touching the sides of said opening 210. There may further be visual markers 130 on the outside surfaces of the main housing 100. In this embodiment, visual markers 130, in the form of screens, are located on the upper section of the surface. In other embodiments, visual markers 130 may be any other type of light emitting means known in the art used to deliver visual information to users. The entry and exit points (110, 120) consist of opening and closing means (115, 125) that may allow the passage of shopping carts when fully opened. In the shown embodiment, curtains are installed at the entry and exit points (110, 120). In other embodiments, any type of door may be installed instead.

The system, requiring a power source to function, may be powered by different power systems (not shown). In an embodiment, the system may be plugged directly to a high-tension source. In another embodiment, the system may have a battery system in order to store energy so that the system may function even when not having an active power source. The battery system may be located anywhere convenient and functional on or in the main housing and its specifications may be configured from anything known in the art. In yet another embodiment, the system may comprise photovoltaic components, such as solar panels, for powering itself. The photovoltaic components may be locating on the outside upper surfaces of the main housing 100. Each power system may be configured to function independently or in conjunction with the other power systems.

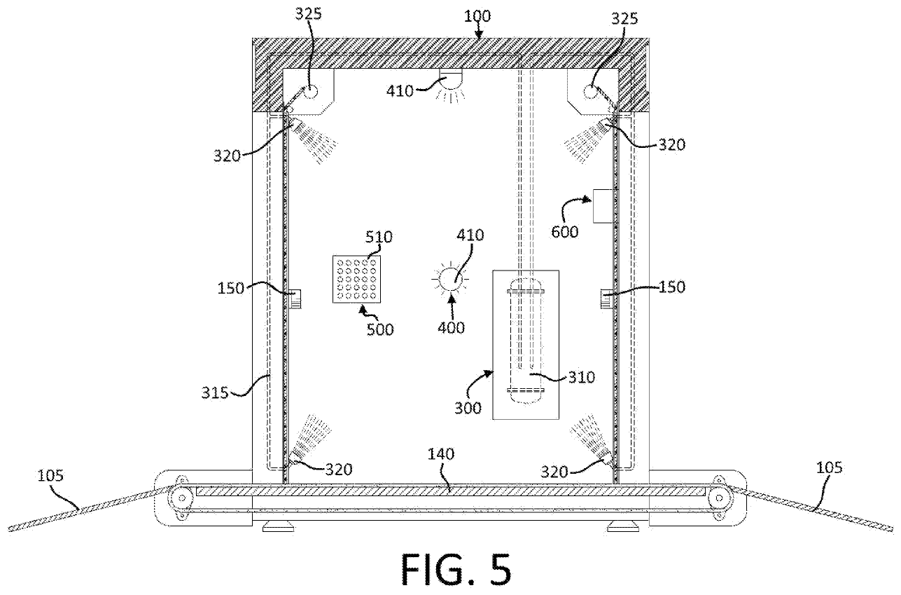

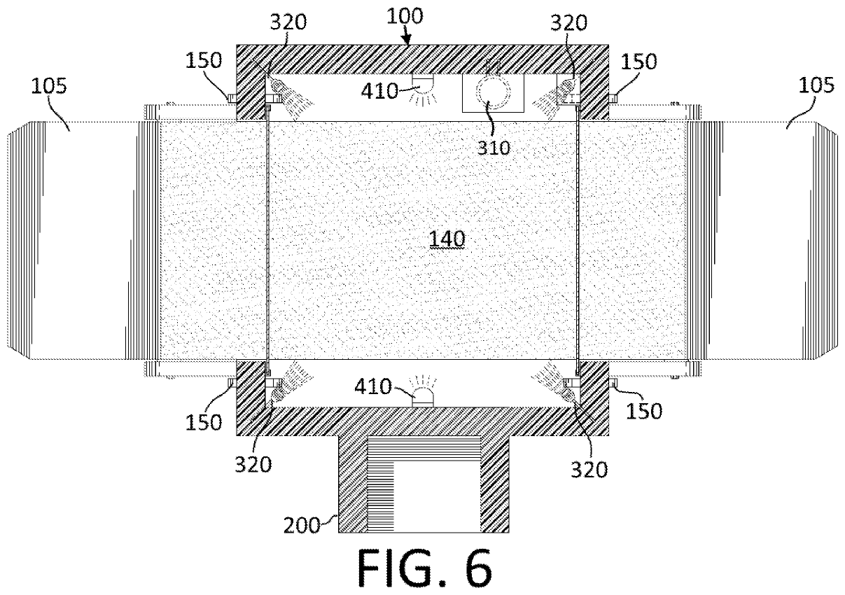

Now referring to FIGS. 5 and 6, the sectional views allow the visualization of the main cleaning and disinfecting components of the system which are: steam system 300, ultraviolet system 400 and microwave system 500. The three systems (300, 400, 500) may be in direct communication with a central computing system 600 and may receive instructions from such computing system. The three systems (300, 400, 500) may thus work simultaneously or in a predetermined or calculated sequence to clean and disinfect shopping carts. Even if the system functions the most effectively when the three systems (300, 400, 500) are functional, each of those systems, independently from the others, may be configured to disinfect shopping carts and reduce germs at a certain percentage even if the other systems are non-functional. The central computing system 600 may have predetermined sequences of operation for the cleaning and disinfecting of shopping carts and may also comprise emergency sequences, also referred to as emergency systems, that may override the other sequences. In some embodiments, the central computing system may be in a protective housing located independently from the rest of the systems. In other embodiments and in order to minimize the possible leaks, the central computing system 600 may be housed within the same protective housing than the microwave source.

The steam system 300 comprises a water tank 310 fluidly connected to at least one spray 320 located inside the main housing 100 by the use of piping 315. The water tank 310 provides pressurized water which turns into steam due to the high pressure and temperature of said water. A heating system, not shown, may be added to help raise the temperature of the water. In a preferred embodiment and in order to be effective against germs, the steam in the system in contact with the objects to disinfect, such as shopping carts, is at a minimal temperature of 60° C., with 90° C. being another effective temperature for disinfection. The at least one spray 320 is oriented to direct the outgoing steam in the direction of shopping carts stationed inside the main housing 100. In some embodiments, steam sprays 320 may comprise mechanisms 325 allowing movement of the spraying direction for better coverage without requiring the addition of new sprays 320. The mechanisms 325 may also be configured to open and close the entry and exit points (110, 120) in certain embodiments. In the embodiment shown, there are eight steam sprays 320, each one located at an inner corner of the main housing 100. The sprays 320 located at the upper corners of the main housing 100 are connected to mechanisms 325 allowing the displacement of the spraying direction. At the bottom of the main housing 100, generally under the conveying system 140, an accumulation receptacle (not shown) may be configured to receive any fluid left in the housing 100, such as water resulting from cooled off steam. The accumulation receptacle may have varying forms, such as a displaceable tray or a simple tub. A fluid discharge system, not shown, may further be installed to allow the exhaust of fluid from the accumulation receptacle or from the system itself. The fluid discharge system may be fluidly connected to the accumulation receptacle or the main housing 100 itself on one hand and to a municipal wastewater system on the other hand in order to effectively exhaust fluid and thus avoid an over-accumulation of said fluid inside the main housing 100. Compared to prior art documents in the same field wherein fluid sprayed over objects to clean is a solution comprising strong chemicals, the present invention does not use chemicals in the fluid. Indeed, the system may use water without any chemical additive. Water is the preferred fluid to be used and, in some embodiments, natural solutes may be added to said water. As such, no significant harm is to be done to the environment when waste water from the system is exhausted into external systems or nature itself.

The ultraviolet system 400 comprises at least one ultraviolet light 410 located inside the main housing 100. The at least one ultraviolet light 410 emits electromagnetic waves in a certain wavelength range that is effective against germs. In one embodiment, the electromagnetic waves may have a range from 100 nm to 280 nm, which has been proven effective against germs. The ultraviolet system's 400 goal is thus to disinfect shopping carts stationed inside the main housing 100 without having any direct contact to said shopping carts. In the embodiment shown in FIGS. 1 to 6, there are three ultraviolet lights 410 located inside the main housing 100 and one ultraviolet light 410 located inside the 200. As mentioned above, the disinfecting box opening 210 allows users to insert their hands inside said box 200 in order to sanitize, and possibly dry, them with ultraviolet light. Having a system for disinfecting users' hands may greatly be effective in reducing or preventing the use and waste of chemicals as only ultraviolet lights are emitted.

The microwave system 500 comprises at least one electromagnetic wave emitter 510. The electromagnetic waves emitted are in the range of microwaves and are used to disinfect shopping carts stationed inside the main housing 100. In one embodiment, the electromagnetic waves may have frequency of 2 to 4 GHz. As seen in FIG. 5, a section of the main housing 100 comprises small holes which is the electromagnetic wave emitter 510. As is known from the prior art, the presence of water droplets attached to a metallic object when microwaves are introduced helps reduces, if not supress, arcing which may otherwise normally occur. Having steam introduced in the main housing 100 by the steam system 300 is thus essential for the use of microwaves without significant arcing.

Due to the use of electromagnetic waves and high temperature fluids, the main housing 100 are preferably hermetically sealed when shopping carts are being treated. Consequentially, the opening and closing means (115, 125) must have hermetic seals, not shown, prohibiting the exhaust of particles or electromagnetic waves from the entry and exit points (110, 120) when the system is in use. Any seal mechanism known in the art may be used to properly seal the opening and closing means (115, 125). It has been proven that the absence of air further helps reduce the arcing that may occur when microwaves are introduced in the main housing 100. Thus, in another embodiment, a valve, not shown, might discharge air present inside the main housing before microwaves are introduced in order to prevent arcing.

At least part of the bottom surface inside of the main housing 100 is a conveying system 140. The conveying system 140 is generally a conveyor and comprises a mechanical or electro-mechanical system moving a conveying belt. In some embodiments, such as the one shown in FIGS. 5 and 6, the conveyor belt spans the distance from the entry to the exit points (110, 120) of the main housing 100 and further continues outside of said compound before being adjacent to the elevation means 105 on each side. In other embodiments, the conveying system 140 replaces the elevating means by extending all the way to the floor at an angle allowing shopping carts to be elevated without requiring manual labour.

The system further comprises a multitude of sensing elements 150. The sensing elements 150 may be located at various locations both inside and outside of the main housing 100. They may be used to detect the presence of objects where their positioning allows. As such, they may be in direct communication with the central computerized system to help control other components and systems when in operation. As seen in FIGS. 1 to 6, the sensing elements 150, which are proximity sensors in this embodiment, are positioned adjacent to the entry and exit points (110, 120) as to assess when shopping carts are passing and/or have passed through said entry and exit points (110, 120). The disinfecting box 200 may further comprise proximity sensors adjacent to the opening 210 to timely activate the ultraviolet light 410 in said disinfecting box 200 when hands are inserted. Other types of sensors may be used, such as temperature sensors, pressure sensors, humidity sensors, etc. Sensing elements 150 may further need specific protection against damaging conditions, especially inside the main housing 100. As such, any protection method known in the art may be used.

As mentioned above, the central computing system 600 may comprise emergency sequences that may override the usual operating sequences. In an embodiment, emergency sequences may be associated with infrared sensors 150 located inside the main housing 100 that may detect the presence and movement of a heat source inside said main housing 100. The heat source may come from a child, an animal or any other living being not supposed to be located in the main housing 100. Thus, when detecting a heat source that is not supposed to be present inside the main housing 100 or movement from said heat source, an emergency sequence may block or terminate any other operating sequences and cause the entry and exit points (110, 120) to open. Emergency sequences may further be configured to be activated when emergency buttons or switches, not shown, are activated. Emergency buttons and/or switches may be located both inside and outside of the main housing 100 and may be activated by users both inside and outside of the main housing 100. For additional safety, at least one camera, not shown, may be located inside the main housing 100 and may project images of what is happening inside said main housing 100 to visual markers 130 or to an external computerized device in connection with the central computing system 600.

A method of cleaning and disinfecting shopping carts is also provided by the present invention. The method uses a cleaning and disinfected system in accordance with the embodiments presented above and comprises the steps of : positioning a shopping cart in connection with a conveying mean so that it may be moved without manual interference, advancing the shopping cart to a first sensing element, opening an entry point, advancing the cart to a stationary position in a main housing, hermetically closing the entry point, activating a steam system, activating a microwave system, activating an ultraviolet system, showing the progress of a cleaning and disinfecting predetermined sequence to users with visual markers, opening an exit point, advancing the cart out of the main housing and closing the exit point once a sensing element located at the exit point does not sense the presence of an object. The method may further comprise discharging microwaves through a holed section of the main housing and discharging fluids accumulated in the main housing through a fluid discharge system. In another aspect of the method, microwaves may be emitted after the activation of the steam system for a predetermined period of time. The period of time may range from a couple seconds to a couple of minutes. In yet another embodiment, the period of time of emitted microwaves being between 30 to 90 seconds. In another embodiment, the method may further comprise discharging air present in the main housing by activating an air valve. In yet another embodiment, the method may comprise cleaning and disinfecting multiple stacked shopping carts instead of one shopping cart only. With safety in mind, the method may also comprise emergency systems overriding the operating sequences when an emergency is detected. It may be appreciated that the method allows for the cleaning and disinfecting of shopping carts without manipulation from users other than at the initial positioning of said shopping carts, thus reducing the chances of users contamination compared to other methods known in the art.

While illustrative and presently preferred embodiment(s) of the invention have been described in detail hereinabove, it is to be understood that the inventive concepts may be otherwise variously embodied and employed and that the appended claims are intended to be construed to include such variations except insofar as limited by the prior art.

1) A system for cleaning and disinfecting at least a shopping cart, the system comprising:
a main housing configured to house the at least one shopping cart and having an entry point and an exit point, the entry and exit points being closable;
a conveying means configured to displace the at least one shopping cart from the entry point to the exit point;
a steam system configured to emit steam in the main housing;
an ultraviolet system configured to emit ultraviolet light in the main housing; and
a microwave system configured to emit microwaves in the main housing.

2) The system of claim 1, the steam system comprising a water tank containing pressurised water and at least one spray fluidly connected to the water tank.

3) The system of claim 2, the steam system further comprising a heating system for heating the pressurised water.

4) The system of claim 1, the steam being at a temperature in a range of 60 to 90° C.

5) The system of claim 1, the emitted ultraviolet light having a wavelength in a range of 100 nm to 280 nm.

6) The system of claim 1, the system further comprising a disinfecting box for washing hands of a user, wherein the ultraviolet system is further configured to emit ultraviolet light in the disinfecting box.

7) The system of claim 1, the emitted microwaves having a frequency in a range of 2 to 4 GHZ.

8) The system of claim 1, the main housing comprising a fluid accumulation receptacle.

9) The system of claim 8, the system further comprising a fluid discharge system for the discharge of accumulated fluids inside the fluid accumulation receptacle.

10) The system of claim 1, the system further comprising visual markers showing progress of the cleaning and disinfecting of the at least one shopping cart.

11) The system of claim 1, the entry and exit points comprising opening and closing means, the closing means hermetically sealing the main housing when closed during the cleaning and disinfecting of the at least one shopping cart.

12) The system of claim 11, the system further comprising an air valve to discharge air present in the main housing.

13) The system of claim 1, the system further comprising an emergency system for stopping the cleaning and disinfecting of the at least one shopping cart.

14) The system of claim 13, the emergency system being in connection with an infrared sensor for detecting unwanted heat in the main housing, the emergency system activated when unwanted heat is detected.

15) A method for cleaning and disinfecting at least one shopping cart, the method comprising the steps of:
positioning a shopping cart in connection with a conveying means;
opening an entry point;
advancing the at least one shopping cart to a stationary position in a main housing;
closing the entry point;
activating a steam system;
activating a microwave system;
activating an ultraviolet system;
opening an exit point;
advancing the at least one shopping cart out of the main housing; and
closing the exit point.

16) The method of claim 15, the method further comprising showing the progress of the cleaning and disinfecting of the at least one shopping cart with visual markers located outside the main housing.

17) The method of claim 15, the method further comprising discharging air present in the main housing by activating an air valve.

18) The method of claim 15, the method further comprising activating the microwave system for a period of 30 to 90 seconds.

19) The method of claim 15, the method further comprising discharging fluids accumulated in the main housing through a fluid discharge system.

20) The method of claim 15, the method further comprising stopping any on-going operation when an emergency system is activated.
